SEQUENCE LISTING (25 sequences)

SEQ ID NO: 1 - DNA, 3664 bp, Homo sapiens
gaattaagcattttagcattctttattaatttttcaaagtcactaggaccaaggataacaattcatcatgtgcatacaaggccattctgtgtttcctactcttgccttgggctcatcattattaatctggaattccatttgttcttcactttttgaatatgtctgtttagttgactgtagtgccactggcaggaccatgtgcccaggaaatccaagactcatatttggacgaaagctatgtccacttttcaactagtacccctacccaaattaccatagcaaccaaaaaattgcagatgcctacattctagaatcatgttctaaagggatgtcatcatttacaaaatgtctttgttgagtctgaatggttcaaacaatagcaaaaaaggattatttctctcttggacatttcaaagtactatgacacaaaatatccaagacttgttatggtgaggagccaagtggaatggaaaggacagctcatcccggcggctgggagtgcatgcacacacatgccccctttttcttgcctactaacaggatctatagaaggcgtacataatgagtatgtaggggacttggctgctttcagttaggaatgagacactgatatggttggaatatagtaagagaaaaagggaggtctttcttaaaaaacggttttgtgtaaaaatagagatggcacttaatggatatcatattagcaggctccctggacaaatacatagagccaaaacttctcatcgattagccacctcttcaagtttaggggttgaaaatctgaaacaactacaaacatggtatctctctgaaaaggagataacgtaaaagttatcacatattaatataatgtgtatgaataaattgacaagctggttagaaattagaaataaaagtcttgaggcaataaaagaggtaataacataggcaaaaagagctcttcttctggagagtggttgtagatggagtaaacaagtttaggtactgaactgagaatagcacatggatagaccaattgtggatgaaggagactaaagagaggtttaacgaatattgaaatgaacctccaggtaggttgtatttattagtttgctgggaacaagctgcttttctctctcctgtgaagcaggaaggcaaatttctagtggctttccaaaggaaatgggaaatctaaggaaatggtttgataccagagtgttctccttaggtttattttaatgatggacttaaagatactttcctatactcatgagctatgttgtctctgatattctttggtatattttaccaaaaagatagaataggtgccacaagtattaaaaattttagactcctcagagcattacaaaaaacaagcacaaaatagaagcctaatatgcagggaaagtcactgaccatgcccttggtactgctgattgtattgcagagcaagagatggaccctgagggtacttgaagccaacaagtttcacttctggaaaaagacttcagaatatgagtttaaaatataaaaagggaatttgagccaagacacaagaacaaactttttttgacaattatatctttattattcctcttacagagctacatttactcttactaagtttcagagtcaggtagtaatttacagtaagactgaattaccatccataacgttagatgtccttattgaaacttcaacatcatttccaaatatcagcattagcattgtgcttgacattcatttaacgaagttactgaaaatctattaagtataagacatcagttatttttaatagaagtttctgaaaacatttcagcaaaatagcctgttgagaaaaatgtgtatgctgaaaaaaaaaaatgaacaaataggaaagcctggttcacaaacaggtgtcagggaaatagacagtacttttatagtaataacataagaacaaacttcttgaaggtaagttttattaaataataggacaacaacaagataaaatgacttcttcctgatatttatatattgattgctggctggtcataagactgtttttaggcaacgtgttttgaaaaaccagaaagtctactaccttgagttttcagccacgtgagaatagcaagattcagtgtttatacttgatagcatcttaattaggcctacaggcctccctttcacataactaccttcaagtttatgacagctcaaactcacaattatcattatggagaagagagaagagttaagctaaaaacagaccactttcagaggacctgaaagcaacgtaatcagtcacctattgccatatacaagccacccccaaacataatgacttaaaacagcgatcatctattattgcttatgagtctctgagtcagctgaacattcctgctgatctgggcttggttaggcttattttagctgtgttcattcttggtctgcagatagctgacaatcacctaggggctgactgtaggcattccagctgagatatgctctctgtgtcttttatcctttagcaggaggaggcttgctcacagggtggttacaggcatccaagagagtcagcataaatgtgaaaagtttccaaaatatcagattcagtcctatgtaatctggtttccattgcattctcttggccagagcaagttgcaagacaagtccaaattcaagaaggtcaagaaatacactccatctccaggtaggagaagctgcaaagaactgtgacaatctatgacaaatagtatgttcaaagggaataatatgggaagatgtgccctccgccaacttctcagggaaaaatacagcttttgtaatatttagtaatatagactgtctaatatttctagagaaatctatgactttgagttgaaatatctgaggccaacactccaagcaattttaaacaagtggtgacagaaattaccagacacacatcaagactcaagtataaagctatacaatttaaggatgctcagcaaatgttactgaattgactgggtagtccctaaagagctgaagaataaaagatgttatgagaaatccaacaataccaaatataaattgcctcaggttctgaaatattcaataaagtattctcactgtagttccttcagcttagctgatttggactttggctgtgaaaacattatcctcagtgtttaaaaggttggaaaattctactgggtctttggcccaacctggaattaaatcctgatgcttagaacctcaaagtctaaaatcttctattgtcactttacagagctattgaaacatattaataaacttgtatcatactgatttgattctaatttttgtgggacattgtttaaaaattgttgaaatgcatatatggaaaattgattttttaagtaaatgtataacttttaaaattgtatcctacatctaactccaaataaaggtttaaaaacaactatgagcaatataagtaatacatttaaaatacatttaagagaaagataaggaaaaaaggaatgactcatgaaggttagtacacaatctatgcatcttgaatatttgcacacttaccaagtatttggctccagggtttctggcagctaatgcaaagagaggaacagaatcaagtttcatggtattatctggtagactgtggaagctatagcatttctgccccctcatgttttcacattcccctttagagaacagcacaata

SEQ ID NO: 2 - DNA, 22 bp, Artificial Sequence
  synthetic primer
actaagaagtgcattagtcggg

SEQ ID NO: 3 - DNA, 20 bp, Artificial Sequence
  synthetic primer
ttcctgtgctctagcttgct

SEQ ID NO: 4 - DNA, 20 bp, Artificial Sequence
  synthetic primer
tgcaaatctatgctgcaaaa

SEQ ID NO: 5 - DNA, 20 bp, Artificial Sequence
  synthetic primer
ggttgcctaatcacgagaaa

SEQ ID NO: 6 - DNA, 25 bp, Artificial Sequence
  synthetic primer
ccaaaggcttggtgatttagtggac

SEQ ID NO: 7 - DNA, 25 bp, Artificial Sequence
  synthetic primer
ctagattgaaggccagaaaacatgc

SEQ ID NO: 8 - DNA, 19 bp, Artificial Sequence
  synthetic primer
aacatcttagggcatcctg

SEQ ID NO: 9 - DNA, 25 bp, Artificial Sequence
  synthetic primer
aatgatttaaaatagattaggagca

SEQ ID NO: 10 - protein, 62 aa, Homo sapiens
MVRSQVEWKGQLIPAAGSACTHMPPFSCLLTGSIEGVHNEASCKTSPNSRRSRNTLHLQRNL

SEQ ID NO: 11 - protein, 45 aa, Homo sapiens
MVRSQVEWKGQLIPAAGSACTHMPPFSCLLTGSIEGVHNEARDGP

SEQ ID NO: 12 - DNA, 189 bp, Homo sapiens
atggtgaggagccaagtggaatggaaaggacagctcatcccggcggctgggagtgcatgcacacacatgccccctttttcttgcctactaacaggatctatagaaggcgtacataatgaagcaagttgcaagacaagtccaaattcaagaaggtcaagaaatacactccatctccagagaaatctatga

SEQ ID NO: 13 - DNA, 138 bp, Homo sapiens
atggtgaggagccaagtggaatggaaaggacagctcatcccggcggctgggagtgcatgcacacacatgccccctttttcttgcctactaacaggatctatagaaggcgtacataatgaagcaagagatggaccctga

SEQ ID NO: 14 - DNA, 20 bp, Artificial  Sequence
  synthetic primer
cccaggaaatccaagactca

SEQ ID NO: 15 - DNA, 21 bp, Artificial  Sequence
  synthetic primer
ctccttcatccacaattggtc

SEQ ID NO: 16 - DNA, 21 bp, Artificial Sequence
  synthetic primer
tcatcgattagccacctcttc

SEQ ID NO: 17 - DNA, 21 bp, Artificial Sequence
  synthetic primer
tgtcaagcacaatgctaatgc

SEQ ID NO: 18 - DNA, 23 bp, Artificial Sequence
  synthetic primer
ggtttgataccagagtgttctcc

SEQ ID NO: 19 - DNA, 21 bp, Artificial Sequence
  synthetic primer
gtcttatgaccagccagcaat

SEQ ID NO: 20 - DNA, 21 bp, Artificial Sequence
  synthetic primer
gcattagcattgtgcttgaca

SEQ ID NO: 21 - DNA, 21 bp, Artificial Sequence
  synthetic primer
ctgactctcttggatgcctgt

SEQ ID NO: 22 - DNA, 23 bp, Artificial Sequence
  synthetic primer
gtcacctattgccatatacaagc

SEQ ID NO: 23 - DNA, 21 bp, Artificial Sequence
  synthetic primer
tgttggcctcagatatttcaa

SEQ ID NO: 24 - DNA, 20 bp, Artificial Sequence
  synthetic primer
gctgcaaagaactgtgacaa

SEQ ID NO: 25 - DNA, 23 bp, Artificial Sequence
  synthetic primer
ccaaatacttggtaagtgtgcaa

(19) United States
(12) Patent Application Publication
(10) Pub. No.: US 2007/0275381 A1
(43) Pub. Date: Nov. 29, 2007

(54) Chromosome 5 Genetic Variants Related To Dyslexia

(75) Inventors: Elliot P. Dawson, Murfreesboro, TN (US); Kristie E. Womble, Franklin, TN (US); John A. Phillips, Franklin, TN (US)

Correspondence Address: SHELDON MAK ROSE & ANDERSON PC, 100 East Corson Street, Third Floor, PASADENA, CA, 91103-3842, US

(73) Assignees: BIOVENTURES, INC., Murfreesboro, TN (US); VANDERBILT UNIVERSITY, Nashville, TN (US)

(21) Appl. No.: 10/578,701

(22) PCT Filed: Nov. 13, 2004
(86) PCT No.: PCT/US04/37587

§ 371 Date: May 7, 2007

Related U.S. Application Data
(60) Provisional application No. 60/520,366, filed on Nov. 14, 2003.

(51) Int. Cl. C12Q 1/68; C07H 21/04

(52) U.S. Cl. 435/6; 434/167; 536/23.1

(57) ABSTRACT

An isolated polynucleotide or genetic material from human Chromosome 5 that indicates the presence of dyslexia or a predisposition to develop dyslexia in the individual from whom the sample was obtained. A method of diagnosing dyslexia or a predisposition to develop dyslexia.

### CROSS-REFERENCE TO RELATED APPLICATIONS

The present application claims the benefit of U.S. Provisional Patent Application Ser. No. 60/520,366, filed Nov. 14, 2003, the contents of which are incorporated in this disclosure by reference in their entirety.

### BACKGROUND

Dyslexia is a specific learning disability that is characterized by difficulty recognizing words accurately or fluently, and by a significantly decreased ability to spell related to a difficulty in phonological processing that are inconsistent with the person's age, background and intelligence level. Dyslexia causes problems in reading comprehension and, thereby, can compromise the affected person's education leading to a reduced level of overall achievement.

Dyslexia affects between 15% and 20% of the population in varying degrees of severity, and is the most common cause of difficulty in reading, writing and spelling among students who receive special education services in the United States. The underlying basis for dyslexia is believed to be neurobiological. Numerous familial studies have indicated an inherited basis for dyslexia. Further, genetic studies have implicated a variety of genomic regions as possibly involved in the transmission of dyslexia, including genomic regions on chromosomes 1p, 2p, 3p, 3q, 4q, 6p21.3, 6q, 8p, 9p, 11p, 13q, 15q, 18p11.2, 18q, 21q, and Xq. Unfortunately, none of the genes implicated in dyslexia to date seems to occur in a significant plurality (greater than 10%) or majority of persons diagnosed with dyslexia. Therefore, the diagnosis of dyslexia can only be made by phonological testing which can only be done after a person has reached a suitable age for such testing.

Treatment of dyslexia generally involves phonological training and remedial assistance to compensate for the difficulties experienced by dyslexics. Early intervention is associated with increased function in adulthood. There is not, however, any specific therapy directed to ameliorating the underlying genetic or biological defect.

Therefore, there remains a need for a genetic test to diagnose dyslexia. Further, there remains a need for a method of treating dyslexia that does not depend upon diagnosing dyslexia through phonological testing. Additionally, there remains a need for a biologically based method of treating dyslexia that involves compensating for the underlying genetic or biological abnormality.

### SUMMARY

According to another embodiment of the present invention, there is provided isolated genetic material from human Chromosome 5 of an individual that indicates the presence of dyslexia or a predisposition to develop dyslexia in the individual from whom the material was obtained, the material comprising an allele of each of at least two microsatellite markers flanking SEQ ID NO: 1 in combination on Chromosome 5: Haplotype #8 the 190,198 microsatellite combination of D5S1487/D5S617; Haplotype #9 the 214,190 microsatellite combination of D5S1487/D5S617; and Haplotype #10 the 214,192 microsatellite combination of D5S1487/D5S617.

According to another embodiment of the present invention, there is provided isolated genetic material from human Chromosome 5 of an individual that indicates the presence of dyslexia or a predisposition to develop dyslexia in the individual from whom the material was obtained. The material comprises, a) isolated genetic material according to claim 1; in combination with either b) an isolated polynucleotide comprising at least about 17 consecutive nucleotides of SEQ ID NO:1 including residue 2285, where residue 2286 has an A to C substitution; or comprising at least about 17 consecutive nucleotides of SEQ ID NO:1 including residue 3281, where residue 3282 has a T to G substitution; or comprising at least about 25 consecutive nucleotides of SEQ ID NO:1 including residue 2285, where residue 2286 has an A to C substitution; or comprising at least about 25 consecutive nucleotides of SEQ ID NO:1 including residue 3281, where residue 3282 has a T to G substitution; or comprising at least about 40 consecutive nucleotides of SEQ ID NO:1 including residue 2285, where residue 2286 has an A to C substitution; or comprising at least about 40 consecutive nucleotides of SEQ ID NO:1 including residue 3281, where residue 3282 has a T to G substitution; or c) isolated genetic material from human Chromosome 5 of an individual that indicates the presence of dyslexia or a predisposition to develop dyslexia in the individual from whom the material was obtained, the material comprising a sufficient portion of SEQ ID NO:1 comprising (Haplotype #1) an A to T substitution at residue 879 and a G to A substitution at residue 2613; or comprising (Haplotype #2) an A to C substitution at residue 424, a C to A substitution at residue 554, a C to T substitution at residue 1346, an A to C substitution at residue 2286, a G to A substitution at residue 2314 and a G to A substitution at residue 2613; or comprising (Haplotype #3) a G to A substitution at residue 1145 and a G to A substitution at residue 2613; or (Haplotype #4) comprising an A to C substitution at residue 424, a C to A substitution at residue 554, a C to T substitution at residue 1346, a G to A substitution at residue 2314, a G to A substitution at residue 2613 and a T to G substitution at residue 3282; or comprising (Haplotype #5) an A to C substitution at residue 424, a C to A substitution at residue 554, an A to T substitution at residue 879, a C to T substitution at residue 1346, a G to A substitution at residue 2314, a G to A substitution at residue 2613 and a T to G substitution at residue 3282; or comprising (Haplotype #6) an A to T substitution at residue 879; or comprising (Haplotype #7) an A to C substitution at residue 2286 and a G to A substitution at residue 2613; where except for these substitutions, residue 424 is A, residue 554 is C, residue 879 is A, residue 985 is C, residue 1145 is G, residue 1346 is C, residue 2275 is A, residue 2286 is A, residue 2314 is G, residue 2453 is C, residue 2613 is G, residue 3282 is T; or d) both b) and c).

According to another embodiment of the present invention, there is provided a method of diagnosing dyslexia or a predisposition to develop dyslexia. The method comprises, a) providing a sample from an individual containing genetic material from Chromosome 5; and b) analyzing the genetic material for the presence of one or more than one of Haplotype #8 through Haplotype #10, or isolated genetic material according to the present invention; where the presence of one or more than one of Haplotype #8 through Haplotype #10 or isolated genetic material indicates a diagnosis of dyslexia or a predisposition to develop dyslexia.

According to another embodiment of the present invention, there is provided a method of the present invention, where the sample is obtained in utero or post-mortem.

According to another embodiment of the present invention, a method additionally comprises administering phonological testing to the individual to confirm the diagnosis of dyslexia.

According to another embodiment of the present invention, a method additionally comprises analyzing genetic material from the individual for the presence of one or more than one genetic marker for dyslexia or for a predisposition to develop dyslexia on a chromosome other than Chromosome 5 to confirm the diagnosis of dyslexia. In one embodiment, the chromosome other than Chromosome 5 is selected from the group consisting of Chromosomes 1p, 2p, 3p, 3q, 4q, 6p21.3, 6q, 8p, 9p, 11p, 13q, 15q, 18p11.2, 18q, 21q, and Xq. In another embodiment, the chromosome other than Chromosome 5 are Chromosomes 6p21.3 and 18p11.2.

According to another embodiment of the present invention, there is provided a method of ameliorating the symptoms of dyslexia or preventing dyslexia in an individual. The method comprises, a) diagnosing dyslexia or a predisposition to develop dyslexia in the individual according to the method of the present invention; and b) treating the individual. In one embodiment, treating the individual comprises administering phonological training to the individual.

According to another embodiment of the present invention, there is provided a method of classifying a dyslexic individual or group of dyslexic individuals. The method comprises, a) diagnosing dyslexia or a predisposition to develop dyslexia in the individual or individuals according to the method of the present invention; and b) assigning a classification to the individual or individuals based on the variant or haplotype identified as a result of the diagnosis.

### DESCRIPTION

According to one embodiment of the present invention, there is identified a group of single nucleotide polymorphisms on Chromosome 5 that are related to developmental dyslexia According to another embodiment of the present invention, there is identified a group of haplotypes that are related to developmental dyslexia. According to another embodiment of the present invention, there is provided a method for diagnosing dyslexia or a predisposition to develop dyslexia. According to another embodiment of the present invention, there is provided a kit for diagnosing dyslexia or a predisposition to develop dyslexia. According to another embodiment of the present invention, there is provided a method of treating or preventing dyslexia that involves compensating for the genetic or biological abnormalities.

As used herein, the term “comprise” and variations of the term, such as “comprising” and “comprises, ” are not intended to exclude other additives, components, integers or steps.

As used herein, the term “dyslexia” refers to a language-based learning disorder that is biological in origin, interferes with the acquisition of print literacy, is characterized by poor single-word decoding and spelling abilities, and is further characterized by a deficit in one or both of phonological awareness (letter/sound association) and phonological manipulation (ability to use individual speech sounds appropriately).

As used herein, the term “dyslexic” refers to an individual who exhibits dyslexia or who has a predisposition to exhibit dyslexia in the absence of treatment to inhibit, prevent, alleviate or reverse dyslexia.

As used herein, the term “aberrant form” refers to a genetic sequence that occurs in a human individual that exhibits dyslexia, where the aberrant form causes a phenotype different from the phenotype of the wild type genetic sequence, or causes a predisposition to develop a phenotype different from the phenotype of the wild type genetic sequence.

As used herein, the term “dyslexia-associated isoform” of a gene refers to an isoform of a gene or genetic haplotype that occurs more commonly in the genome of one group of human individuals that exhibit dyslexia than in the genome of another group of human individuals that do not exhibit dyslexia, wherein both groups of individuals are part of the same genealogically-related cohort. It is understood that different cohorts can exhibit different dyslexia-associated isoforms.

As used herein, the term “haplotype” refers to the specific pattern and order of alleles on a chromosome.

As used herein, the term “dyslexic-associated haplotypes” refers to one or more than one genetic variant on the same chromosomal segment of a single chromosome that occurs at a higher frequency in dyslexics than in non-dyslexics.

As used herein, the term “phenotype” refers to the structural and functional properties of an organism which results both from its genotype and its environment.

As used herein, the term “genetic marker” is a polynucleotide sequence within the human genome whose location can be physically or genetically identified with respect to its position relative to other genomic features, and which can be used to demonstrate the heritability or association of a trait.

As used herein, the term “sufficient portion” refers to a polynucleotide having a continuous portion of a sequence to include both identified residues. For example, “a sufficient portion of SEQ ID NO: 1 comprising (Haplotype #1)” means a polynucleotide derived from SEQ ID NO:1, that comprises enough of the nucleotides of SEQ ID NO:1 to identify the variant present at both residue 879 and residue 2613.

According to one embodiment of the present invention, there is identified a group of single nucleotide polymorphisms on Chromosome 5 that are related to developmental dyslexia. Chromosome 5, and in particular, the region on Chromosome 5 5q14.3 or from about Chr5:82,800,001-91,900,000 bases on the strand of human Chromosome 5 (as indicated in May 2004 freeze, UCSC Genome Browser http://genome.ucsc.edu/), and in particular the polynucleotide SNAJA, SEQ ID NO:1, has not been previously known to contain a genetic region harboring a gene or functional genetic element contributing to developmental dyslexia, or that occur at higher frequencies in dyslexics than in non-dyslexics. According to another embodiment of the present invention, there is identified a group of haplotypes on Chromosome 5 that are related to developmental dyslexia. There have not been any previously known haplotypes or combinations of loci on Chromosome 5 contributing to developmental dyslexia, or that occur at higher frequencies in dyslexics than in non-dyslexics.

These aberrant forms appear to occur in one or more than one gene that encodes components of a neuronal development pathway involving genes on other chromosomes. The aberrant forms of these genes include forms in which the sequence of the encoded gene product is altered, and include forms in which genomic regions that, individually or jointly, affect the level of expression, the timing, duration and sites of expression of the gene product are altered. In aberrant forms, the one or more than one altered gene alters the normal functionality of the gene product, such as for example, by decreasing or eliminating the gene product or its function, thereby leading to aberrant neuronal development or function and the occurrence of a dyslexic phenotype. Additionally, the sites of expression, the duration of expression and other aspects of expression critical to the normal function of the gene products of the pathway are altered in such a manner that normal function is affected adversely or deleteriously, resulting in dyslexia. Different dyslexia-associated isoforms of the genes of this pathway can affect gene expression levels differently, leading to differences in the severity and characteristics of the dyslexic phenotypes between dyslexics so affected.

The occurrence of any aberrant form of the present invention indicates that the individual having the aberrant form is at greater risk for exhibiting dyslexia, is at a greater risk for exhibiting more severe dyslexia, or both, than an otherwise identical individual in whose genome the aberrant form does not occur. Similarly, occurrence of two copies of the aberrant form or copies of two aberrant forms in an individual indicates that the individual is at greater risk for exhibiting dyslexia, is at a greater risk for exhibiting more severe dyslexia, or both, than an otherwise identical individual whose genome contains only one copy of an aberrant form.

As will be understood by those with skill in the art with reference to this disclosure, the frequency and occurrence of specific combinations of microsatellite markers exhibit patterns of allele sharing between related dyslexics, that are distinguishable from their non-dyslexic relatives, indicating a specific pattern which is inherited by dyslexics from dyslexic ancestors and which is distinguishable from patterns of the four microsatellite markers inherited by non-dyslexia relatives. The four microsatellite markers are: D5S617 (also known as HS190XC11, AFM190XC11), GenBank accession no. Z23455; D5S428 (also known as AFM238XF4, RH15299, RH9585, HS238XF4), GenBank accession no. Z17072; D5S1487 (also known as GATA26G01, GATA-D5S1487, D5S2850, D5S1487.P9282, G00-365-187), GenBank accession no. G09394; and D5S1459 (also known as GATA23G12, CHLC.31845, CHLC.GATA23G12.31845, GATA-D5S1459, RH59771, RH6192, CHLC.GATA23G12.P9103, G00-364-223), GenBank accession no. G08434. This pattern of inheritance and transmission in the related dyslexics, which is absent in the related non-dyslexics, indicates the presence of an aberrant form at one or more than one locus which contributes to dyslexia contained within the genetic interval on Chromosome 5 defined by this set of four microsatellite markers as shown in Table 1. That is, the related dyslexics share a common haplotype distinct from related non-dyslexics.

TABLE 1

Sequences and Amplicons of Microsatellite
Marker Primers

| | | | AMPLICON |
|---|---|---|---|
| PRIMER NAME | ORIENTATION | PRIMER SEQUENCE | SIZE |
| D5S1487 F01 HEX | FORWARD | SEQ ID NO:2 | 194-222 |
| | | ACTAAGAAGTGCATTAGTCGGG | |
| D5S1487 R01 | REVERSE | SEQ ID NO:3 | 194-222 |
| | | TTCCTGTGCTCTAGCTTGCT | |
| D5S1459 F01 FAM | FORWARD | SEQ ID NO:4 | 90-110 |
| | | TGCAAATCTATGCTGCAAAA | |
| D5S1459 R01 | REVERSE | SEQ ID NO:5 | 90-110 |
| | | GGTTGCCTAATCACGAGAAA | |
| D5S617 F01 FAM | FORWARD | SEQ ID NO:6 | 171-203 |
| | | CCAAAGGCTTGGTGATTTAGTGGAC | |
| D5S617 R01 | REVERSE | SEQ ID NO:7 | 171-203 |
| | | CTAGATTGAAGGCCAGAAAACATGC | |
| D5S428 F01 | FORWARD | SEQ ID NO:8 | 241-255 |
| | | AACATCTTAGGGCATCCTG | |
| D5S428 R01 FAM | REVERSE | SEQ ID NO:9 | 241-255 |
| | | AATGATTTAAAATAGATTAGGAGCA | |

Fam=Fluorescein and Hex=Hexacholro-fluorescein Expected amplicon sizes in bp are based on predicted mobility and would be expected to vary depending upon analytical method and analysis platform, as will be understood by those with skill in the art with reference to this disclosure.

When non-related individuals are tested for dyslexia by the assessment method described herein, two subgroups are identified. The first subgroup has dyslexia or dyslexia with compensation and the second subgroup is non-dyslexia. When the distribution, frequency, and allele sharing of the four markers described above is measured, there is a definite pattern of allele sharing occurring at a higher frequency in the first dyslexic subgroup than in the second non-dyslexic subgroup. This relationship indicates that certain alleles, and their patterns, combinations, and frequencies can be used to identify a subset of dyslexics having a variant genetic element within the segment 5q14.3 between microsatellites D521487 and D5S617 contributing to the genetic susceptibility and the consequent dyslexia phenotype within members of this subgroup.

Additionally, one gene, SNAJA, SEQ ID NO:1, which is expressed in the human brain and in particular in the hippocampus, is found within the region of human Chromosome 5 bounded by the microsatellites D521487 and D5S617. SNAJA, SEQ ID NO:1, is located between two microsatellite markers, D5S1487 and D5S617.

This gene is SNAJA, SEQ ID NO:1: gaattaagcattttagcattctttattaatttttcaaagtcactaggaccaaggataacaattcatcatgtgcatacaaggccattctgtgtttccta ctcttgccttgggctcatcattattaatctggaattccatttgttcttcactttttgaatatgtctgtttagttgactgtagtgccactggcaggacca tgtgcccaggaaatccaagactcatatttggacgaaagctatgtccacttttcaactagtacccctacccaaattaccatagcaaccaaaaaa ttgcagatgcctacattctaGAATCATGTTCTAAAGGGATGTCATCATTTACAAAATGTCTTTG TTGAGTCTGAATGGTTCAAACAATAGCAAAAAAGGATTATTTCTCTCTTGGACATT TCAAAGTACTATGACACAAAATATCCAAGACTTGTTATGGTGAGGAGCCAAGTGG AATGGAAAGGACAGCTCATCCCGGCGGCTGGGAGTGCATGCACACACATGCCCCC TTTTTCTTGCCTACTAACAGGATCTATAGAAGGCGTACATAATGAgtatgtaggggacttggc tgctttcagttaggaatgagacactgatatggttggaatatagtaagagaaaaagggaggtctttcttaaaaaacggttttgtgtaaaaataga gatggcacttaatggatatcatattagcaggctccctggacaaatacatagagccaaaacttctcatcgattagccacctcttcaagtttagg ggttgaaaatctgaaacaactacaaacatggtatctctctgaaaaggagataacgtaaaagttatcacatattaatataatgtgtatgaataaa ttgacaagctggttAgaaattagaaataaaagtcttgaggcaataaaagaggtaataacataggcaaaaagagctcttcttctggagagtg gttgtagatggagtaaacaagtttaggtactgaactgagaatagcacatggatagaccaattgtggatagaaggagactaaagagaggttta acgaatattgaaagaacctccaggataggttgtatttattagtttgctgggaacaagctgcttttctctctcctgtgaagcaggaaggcaaattt ctagtgGctttccaaaggaaatggaaatctaaggaaatggtttgataccagagtgttctccttaggtttattttaatgatggacttaaagata ctttcctatactcatgagctatgttgtctctgatattctttggtatattttaccaaaagatagaataggtgccacaagtattaaaattttagactc ctcagagcattaCaaaaaacaagcacaaaatagaagcctaatatgcagggaaagtcactgaccatgcccttggtactgctgattgtattgc agAGCAAGAGATGGACCCTGAGGGTACTTGAAGCCAACAAGTTTCACTTCTGGAA AAAGACTTCAGAATATGAGTTTAAAATATAAAAAGGGAATTTGAGCCAAGACACA AGAACAAACTTTTTTTGACAATTATATCTTTATTATTCCTCTTACAGAGCTACATT TACTCTTACTAAGTTTCAGAGTCAGGTAGTAATTTACAGTAAGACTGAATTACCAT CCATAACGTTAGATGTCCTTATTGAAACTTCAACATCATTTCCAAATATCAGCATT AGCATTGTGCTTGACATTCATTTAACGAAGTTACTGAAAATCTATTAAGTATAAGA CATCAGTTATTTTTAATAGAAGTTTCTGAAAACATTTCAGCAAAATAGCCTGTTGA GAAAAATGTGTATGCTGAAAAAAAAAAATGAACAAATAGGAAAGCCTGGTTCAC AAACAGGTGTCAGGGAAATAGACAGTACTTTTATAGTAATAACATAAGAACAAAC TTCTTGAAGGTAAGTTTTATTAAATAATAGGACAACAACAAGATAAAATGACTTC TTCCTGATATTTATATATTGATTGCTGGCTGGTCATAAGACTGTTTTTAGGCAACG TGTTTTGAAAAACCAGAAAGTCTACTACCTTGAGTTTTCAGCCACGTGAGAATAG CAAGATTCAGTGTTTATACTTGATAGCATCTTAATTAGGCCTACAGGCCTCCCTTT CACATAACTACCTTCAAGTTTATGACAGCTCAAACTCACAATTATCATTATGGAGA AGAGAGAAGAGTTAAGCTAAAAACAGACCACTTTCAGAGGACCTGAAAGCAACG TAATCAGTCACCTATTGCCATATACAAGCCACCCCCAAACATAATGACTTAAAAC AGCGATCATCTATTATTGCTTATGAGTCTCTGAGTCAGCTGAACATTCCTGCTGAT CTGGGCTTGGTTAGGCTTATTTTAGCTGTGTTCATTCTTGGTCTGCAGATAGCTGA CAATCACCTAGGGGCTGACTGTAGGCATTCCAGCTGAGATATGCTCTCTGTGTCTT TTATCCTTTAGCAGGAGGAGGCTTGCTCACAGGGTGGTTACAGGCATCCAAGAGA GTCAGCATAAATGTGAAAAGTTTCCAAAATATCAGATTCAGTCCTATGTAATCTG GTTTCCATTGCATTCTCTTGGCCAGAGCAAGTTGCAAGACAAGTCCAAATTCAAG AAGGTCAAGAAATACACTCCATCTCCAGGTAGGAGAAGCTGCAAAGAACTGTGAC AATCTATGACAAATAGTATGTTCAAAGGGAATAATATGGGAAGATGTGCCCTCCG CCAACTTCTCAGGGAAAAATACAGCTTTTGTAATATTTAGTAATATAGACTGTCTA ATATTTCTAGAGAAATCTATGACTTTGAGTTGAAATATCTGAGGCCAACACTCCA AGCAATTTTAAACAAGTGGTGACAGAAATTACCAGACACACATCAAGACTCAAGT ATAAAGCTATACAATTTAAGGATGCTCAGCAAATGTTACTGAATTGACTGGGTAG TCCCTAAAGAGCTGAAGAATAAAAGATGTTATGAGAAATCCAACAATACCAAATA TAAATTGCCTCAGGTTCTGAAATATTCAATAAAGTATTCTCACTGTAGTTCCTTCA GCTTAGCTGATTTGGACTTTGGCTGTGAAAACATTATCCTCAGTGTTTAAAAGGTT GGAAAATTCTACTGGGTCTTTGGCCCAACCTGGAATTAAATCCTGATGCTTAGAA CCTCAAAGTCTAAAATCTTCTATTGTCACTTTACAGAGCTATTGAAACATATTAAT AAACTTGTATCATACTGatttgattctaatttttgtgggacattgtttaaaaattgttgaaatgcatatatggaaaattgatttttta agtaaatgtataacttttaaaattgtatcctacatctaactccaaataaaggtttaaaaacaactatgagcaatataagtaatacatttaaaataca tttaagagaaagataaggaaaaaaggaatgactcatgaaggttagtacacaatctatgcatcttgaatatttgcacacttaccaagtatttggc tccagggtttctggcagctaatgcaaagagaggaacagaatcaagtttcatggtattatctggtagactgtggaagctatagcatttctgccc cctcatgttttcacattcccctttagagaacagcacaata has two alternatively spliced forms SNAJA.a, (http://www.ncbi.nih.gov/IEB/Research/Acembly/av.cgi?db=human&1=snaja.aDec03) and SNAJA.b,. (http://www.ncbi.nih.gov/IEB/Research/Acembly/av.cgi?db=human&1=snaja.bDec03), both located between the two microsatellite markers, D5S1487 and D5S617 on Chromosome 5.

In one embodiment, the present invention is a gene product of SNAJA, SEQ ID NO:1. One gene product of SNAJA, SEQ ID NO:1, is the protein snaja a, peptide H5C7619.2, SEQ ID NO:10:
MVRSQVEWKGQLIPAAGSACTHMPPFSCLLTGSIEGVHNEASCKTSPNSRRSRNTLHL QRNL;
the other gene product of SNAJA, SEQ ID NO:1, is the protein snaja b, peptide H5C7619.1;
SEQ ID NO:11:
MVRSQVEWKGQLIPAAGSACTHMPPFSCLLTGSIE GVHNEARDGP;
   however, as used in this disclosure, the term “gene product” includes “conservative substitutions” where an amino acid is substituted for another amino acid that has similar properties, such that one skilled in the art of peptide chemistry would expect the secondary structure and hydropathic nature of the polypeptide to be substantially unchanged. A conservative amino acid substitution occurs when one amino acid residue is replaced with another that has a similar side chain. Amino acid residues having similar side chains are known in the art and include families with basic side chains (e.g., lysine (Lys/K), arginine (Arg/R), histidine (His/H)), acidic side chains (e.g., aspartic acid (Asp/D), glutamic acid (Glu/E)), uncharged polar side chains (e.g., glycine (Gly/G), asparagine (Asn/N), glutamine (Gln/Q), serine (Ser/S), threonine (Thr/T), tyrosine (Tyr/Y), cysteine (Cys/C)), nonpolar side chains (e.g., alanine (Ala/A), valine (Val/V), leucine (Leu/L), isoleucine (Ile/I), proline (Pro/P), phenylalanine (Phe/F), methionine (Met/M), tryptophan (Trp/W)), beta?branched side chains (e.g., threonine (Thr/T), valine (ValV), isoleucine (Ile/I)) and aromatic side chains (e.g., tyrosine (Tyr/Y), phenylalanine (Phe/F), tryptophan (Trp/W), histidine (His/H)).

In another embodiment, the present invention is a coding region of a cDNA that encodes a gene product of SNAJA, SEQ ID NO:1. One example of a coding region of a cDNA of the present invention is SEQ ID NO:12:

ATGGTGAGGAGCCAAGTGGAATGGAAAGGACAGCTCATCCCGGCGGCTGG
GAGTGCATGCACACACATGCCCCCTTTTTCTTGCCTACTAACAGGATCTA
TAGAAGGCGTACATAATGAAGCAAGTTGCAAGACAAGTCCAAATTCAAGA
AGGTCAAGAAATACACTCCATCTCCAGAGAAATCTAtga.

Another example of coding region of a cDNA of the present invention is SEQ ID NO:13: ATGGTGAGGAGCCAAGTGGAATGGAAAGGACAGCTCATCCCGGCGGCTGGGAGT GCATGCACACACATGCCCCCTTTTTCTTGCCTACTAACAGGATCTATAGAAGGCGT ACATAATGAAGCAAGAGATGGACCCtga. As will be understood by those with skill in the art with reference to this disclosure, the term “coding region of a cDNA” or “cDNA that encodes a gene product of SNAJA, SEQ ID NO: 1” or equivalent language includes conservative variants that do not effect the gene product amino acid sequence.

When SNAJA, SEQ ID NO:1, is amplified from genomic DNA and sequenced using the primers in Table 2 for SNAJA, particular variants within the regions covered by the primer sets can be detected. The primers in Table 2 were selected from the interval described above for 5q14.3 or from about Chr5:86,078,076 through 86,081,739, and sequence specificity was verified by alignment and performance of BLAST and BLAT (http://genome.ucsc.edu/cgi-bin/hgBlat?hgsid=36344426) searches. Further, when these variants are analyzed to determine the haplotypic relationships of the variants, for example, by cloning and sequencing to ascertain the cis or trans relationship and distribution of the detected variants or by statistical inference, certain haplotypes are observed to occur at higher frequencies in the dyslexic group than in the non-dyslexic group. When the combination of the occurrence of genetic variants as haplotypes was determined, the frequency of these haplotypes, and the distribution of haplotypes for the gene SNAJA was analyzed for occurrence in dyslexic samples compared to non-dyslexic samples, a set or sets of haplotypes was found to occur more frequently or exclusively in dyslexics indicating an underlying genetic contribution to dyslexia of the haplotypes and consequently the manifestation and observation of the dyslexic phenotype.

A listing of primers suitable to amplify SNAJA, SEQ ID NO:1, by PCR is set forth in Table 2. The listing of primers for SNAJA (Dys Est) was derived from a reference sequence on Chr 5:86230352-86234015 (April 2003 freeze of UCSC genome browser or Chr5:86078076-86081739 May 2004 freeze, UCSC Genome Browser http:///genome.ucsc.edu).

TABLE 2

Sequences and Amplicons of the
SNAJA (Dys EST) Primers

| | | | Amplicon |
|---|---|---|---|
| Name | Orientation | Primer Sequence | Size |
| DYS EST REG1 F01 | FORWARD | SEQ ID NO:14 | 830 |
| | | CCCAGGAAATCCAAGACTCA | |
| DYS EST REG1 R01 | REVERSE | SEQ ID NO:15 | 830 |
| | | CTCCTTCATCCACAATTGGTC | |
| DYS EST REG2 F01 | FORWARD | SEQ ID NO:16 | 978 |
| | | TCATCGATTAGCCACCTCTTC | |
| DYS EST REG2 R01 | REVERSE | SEQ ID NO:17 | 978 |
| | | TGTCAAGCACAATGCTAATGC | |
| DYS EST REG3 F01 | FORWARD | SEQ ID NO:18 | 839 |
| | | GGTTTGATACCAGAGTGTTGTCC | |
| DYS EST REG3 R01 | REVERSE | SEQ ID NO:19 | 839 |
| | | GTCTTATGACCAGCCAGCAAT | |
| DYS EST REG4 F01 | FORWARD | SEQ ID NO:20 | 841 |
| | | GCATTAGCATTGTGCTTGACA | |
| DYS EST REG4 R01 | REVERSE | SEQ ID NO:21 | 841 |
| | | CTGACTCTCTTGGATGCCTGT | |
| DYS EST REG5 F01 | FORWARD | SEQ ID NO:22 | 598 |
| | | GTCACCTATTGCCATATACAAGC | |
| DYS EST REG5 R01 | REVERSE | SEQ ID NO:23 | 598 |
| | | TGTTGGCCTCAGATATTTCAA | |
| DYS EST REG6 F01 | FORWARD | SEQ ID NO:24 | 850 |
| | | GCTGCAAAGAACTGTGACAA | |
| DYS EST REG6 R01 | REVERSE | SEQ ID NO:25 | 850 |
| | | CCAAATACTTGGTAAGTGTGCAA | |

The Examples disclose methods that were used to identify human genes and haplotypes (SNAJA) on chromosome 5 that are associated with occurrence of dyslexia in individuals. A similar method can be used to identify other human genes associated with occurrence of dyslexia. In this method, a chromosomal region that includes multiple genes and that is associated with occurrence of dyslexia in a plurality of humans (who can, but need not, be genealogically related) is identified. Thereafter, the genes that occur in the dyslexia-linked chromosomal region are examined. Occurrence in that region of a gene known or believed to encode a product that modulates neuronal function or development or both (e.g., a gene or gene product that interacts with the SNAJA protein or its isoforms) is an indication that the gene is linked with dyslexia. If not known, the purported effect of the identified gene on neuronal development can be tested using one of the models disclosed herein.

The present invention is now disclosed with reference to the following Examples. These Examples are provided for the purpose of illustration only and the invention is not limited to these Examples, but rather encompasses all variations which are evident as a result of this disclosure.

### EXAMPLE 1

### Determination of a Specific Dyslema-Related Haplotype and Single Nucleotide Polymorphisms in a Related Population

A) Selection of a Kindred Family Subject Cohort Containing Individuals Afflicted with Dyslexia

The proband was identified from an individual whose dyslexic status was identified within the database of dyslexic subjects at the Tennessee Center for the Study and Treatment of Dyslexia (The Center). Additional kindred of the proband were recruited and evaluated with respect to their dyslexic status by the Head of The Center. Dyslexic status of each participating subject from the kindred was determined using published and publicly available methods employed by The Center. Although consent to analyze the samples was granted, apart from the phenotypic dyslexic status of the individuals from which the samples were derived, no additional information, such as endophenotype or chromosomal loci or potential candidate genes, were provided.

The dyslexic individuals generally conformed to the generally accepted standard for diagnosis of dyslexia as described in the Diagnostic and Statistical Manual of Mental Disorders—Fourth Edition (DSM-IV), (published by the American Psychiatric Association, Washington D.C., 1994), code 315.00 for Reading Disorder, as follows:

’Dyslexia:
Developmental Reading Disorder (DRD) or Dyslexia is a defect of the brain's higher cortical processing of symbols. Children with DRD may have trouble rhyming and separating the sounds in spoken words.
As measured by a standardized tests, the patient's ability to read (accuracy or comprehension) is substantially less than you would expect considering age, intelligence and education. This deficiency materially impedes academic achievement or daily living.

Associated Features:
1. Deficits in Expressive Language and Speech Discrimination are usually present.
2. Expressive Writing Disorder is often present.
3. Visual Perceptual Deficits are seen in only about 10% of cases.
4. Disruptive Behavior Disorders may also be present, particularly in older children and adolescents.
Dyslexia is a specific learning disability that is neurological in origin. It is characterized by difficulties with accurate and/or fluent word recognition and by poor spelling abilities. These difficulties typically result from a deficit in the phonological component of language that is often unexpected in relation to other cognitive abilities. Secondary consequences may include problems in reading comprehension and reduced reading experience that can impede growth of vocabulary and background knowledge.

Dyslexia in the Pre-School Child:
1. Delay or difficulty in development of clear speech and a tendency to jumble words and phrases over some time.
2. Difficulty with dressing efficiently, tying shoe laces, and putting clothes on in the right order.
3. Unusual clumsiness and difficulty with co-ordination.
4. Poor concentration such as when stories are read to them.
5. Ambidextrous or left-handedness.
6. Inability to associate sounds with words.
7. Inability to appreciate rhyme.
8. Family history of similar difficulties.'
As used in this example, individuals were identified as having phonological dyslexia using the following diagnostic criteria:
1. Average or above spatial/reasoning abilities.
2. Nonsense word score substantially below real words score.
3. Spelling scores about equal to nonsense word scores.
4. Very low scores on phoneme segmenting, blending, phoneme manipulation, and auditory discrimination.
5. Personal history.
6. Rapid naming scores were low average or above average.

Individuals were identified as having compensated phonological dyslexia using the following diagnostic criteria:
1. Average or above spatial/reasoning abilities.
2. Nonsense word score substantially lower than real words score.
3. Spelling scores about equal to real word scores.
4. Weak phoneme segmenting scores.
5. Personal history.
6. Rapid naming scores ranged from below average to superior.

Individuals were identified as non-dyslexic using the following diagnostic criteria:
1. Average or above spatial/reasoning abilities.
2. Word reading (real and nonsense) and spelling scores equal or superior to spatial/reasoning scores.
3. Strong phoneme segmenting and manipulation scores.
4. Personal history.
5. Rapid naming scores were average or above.
   B) Sample Preparation

a) Genome-Wide Screening of Individuals Afflicted with Dyslexia

Genomic DNA samples were obtained from each individual of the cohort identified in Example 1 from peripheral blood samples or from buccal swabs. A full genome scan was performed for each of the individuals of the cohort. The methods used to perform this scan were as follows.

DNA was isolated from subject samples using commercially available kits and instruments, such as the MagNA Pure™ DNA isolation instrument (Roche Diagnostics Corporation; Indianapolis, Ind. US), the PUREGENE® DNA isolation kit (Gentra Systems; Minneapolis, Minn. US), or the QIAmp™ (Qiagen Sciences, Inc.; Germantown, Md. US). Saliva samples were collected from subjects using buccal swabs for collection and were processed similarly to obtain genomic DNA when blood could not be obtained.

Genomic DNA samples were diluted to a final concentration of 2 ng per microliter using 0.1×TE (prepared from 10×TE=100 mM Tris hydoxyaminomethane, 10 mM Na₂EDTA at pH 7.4). Samples were dispensed into 96 well PCR plates aliquots using an automated pipettor, dried at 60° C., and placed at 4° C. until ready for use.

C) Genotyping

PCR amplification was performed in 10 microliter reaction volumes using the ABI PRISM® 10 centimorgan resolution Linkage Mapping Set Version 2.5 (Applied Biosystems, Inc., Foster City, Calif. US, product number LMS-MD-10) of fluorescently labeled microsatellite markers, which includes markers that span the human autosomes. Cycling conditions were consistent with those recommended by the manufacturer. Following amplification, the reaction products for each panel were combined consistent with manufacturer's recommendations. An aliquot of 2.0 ul of the pooled panel reactions was added to 3.5 ul de-ionized formamide containing 4 nanomoles of tetramethyl-rhodamine-labeled HD400 (Applied Biosystems, Inc.). This mix was prepared for each sample followed by denaturation at 95° C. for 5 min and rapid cooling to 4° C. Aliquots of 1.2 microliters of the mix for the individual sanples were loaded into the teeth of a 48-lane gel loading comb (The Gel Company; San Francisco, Calif. US). Samples were electrophoresed and detected using an ABI PRISM® (Applied Biosystems, Inc., model 377 DNA sequencing apparatus) following the manufacturer's recommendations.

Data was processed using GENESCAN® (Applied Biosystems, Inc.) Software for initial sizing of the MD-10 panel PCR products for each sample. Sizing and stutter bands were corrected using TEMPLATE® and GENOTYPER® software (Applied Biosystems, Inc.), using automated allele calling. These data were exported to a computer spreadsheet (EXCEL®, Microsoft Corporation; Redmond, Wash. US). Fragment sizes were rounded to the nearest whole number consistent with specific panel members (i.e., to even whole numbers if the marker was an even numbered series, for example 121.5 to 122 if marker was 120, 122, 124, etc., or to odd whole numbers if the marker was an odd numbered series, for example 121.5 to 121 if panel is 119, 121, 123, etc.).

All 382 autosomal markers were genotyped, and 18 markers on the X chromosome were excluded, because an autosomal dominant mode of inheritance was observed in the kindred disclosed in this Example. Linkage analysis identified which markers co-segregated with the dyslexia phenotype for members of the kindred disclosed in this Example. These methods were used to generate LOD scores assuming an autosomal dominant model with a disease allele frequency of 0.001.

Using these methods, the peak LOD score was 2.9 for the interval containing markers D5S641-D5S428-D5S644-D5S433. When additional samples were genotyped and included in the linkage analysis, the peak LOD scores were 1.6, 2.5, 3.2 and 3.2, respectively, for the above markers. The linkage results indicated that the interval spanned by these markers contains the locus to which dyslexia can be attributed in affected individuals of the kindred described in this Example.

Because the chromosomal interval spanned by markers D5S641-D5S428-D5S644-D5S433 is larger than 20 million base pairs and contains more than 110 genes, additional relatives in the kindred described in this Example were genotyped using an additional marker (D5S617) between D5S641 and D5S428 which yielded a peak LOD score of 3.4. Analysis of haplotypes revealed recombinant meioses that narrowed the critical interval to less than 5 million base pairs at chromosomal location 5q14.3.

D) Haplotype Refinement

Using the data obtained using D5S428 and D5S617 an additional polymorphic marker D5S1487 was used to further develop the haplotype. This marker was chosen due to findings that a recombination event was detected which excluded the region telomeric to a position between D5S617 and D5S644 as containing a segment associated with dyslexia, consequently the additional marker was used. Primers utilized for performing the haplotype are listed in Table 1.

E) Candidate Gene Screening

Several known, partial and hypothetical segments of genes in the critical interval were screened by PCR and sequencing, in particular MEF2C, CCNH, RASA1, COX7C, WAMI and SNAJA. All genes except SNAJA were excluded because of discontinuity in haplotypes between individuals inconsistent with an autosomal dominant model for dyslexia transmission.

F) Primer Selection and Optimization for SNAJA

Because SNAJA is within the critical interval it was selected for primer design to provide overlapping fragments which could be amplified by PCR and subsequently sequenced. Primers were selected using a combination of software (Primer 3, http://www-genome.wi.mit.edu/genome_software/other/primer3.html) and manual selection as appropriate using SEQ ID NO: 1 and the selected primers are shown in Table 2.

Primers were optimized using 10 ng of human genomic DNA (Roche Diagnostics Corporation), 10 pmoles each of forward and reverse primers, 10% 10×PCR buffer, 2 mM MgCl₂, 2% Dimethyl Sulfoxide, 5 mM DTT, 200 uM of each dNTP, and 0.625 units of TaqGold (PE Biosystems; Foster City, Calif. US) with 1% Pfu Turbo Hotstart (Stratagene; La Jolla, Calif. US) in a total volume of 20 ul per reaction.

Reaction components were assembled in an MJ Research 96-well Multiplate and briefly pulsed in a centrifuge to mix components. The plate was sealed with Microseal “A” Film, and cycling was performed on an MJ Research Thermalcycler using calculated control and a 50-72° C. gradient, and cycling was performed on an MJ Research Thermalcycler using calculated control and heated lid with cycles consisting of 95° C. for 12 min followed by 35 cycles consisting of 95° for 30 seconds, 50-72° C. gradient for 20 seconds, 72° C. for 40 seconds with a final extension of 72° C. for 6 min. Electrophoresis to assess quality of the amplicons was performed using 2 ul of each product and 5 ul BioMarker DNA sizing standard (BioVentures, Inc; Murfreesboro, Tenn. US) run on precast Nuseive/GTG 3:1 agarose gels containing ethidium bromide (BMA CORP; Rockland, Me. US.) Optimal annealing temperature for each primer pair was selected.

TABLE 3
0ptimal Annealing Temperatures and Extension Times of the Primer
Pairs Based upon Gradient Cycling and Product Length

| Primer pair | Optimal Annealing Temp | Extension Time |
|---|---|---|
| DYS EST REG1 F01 - R01 | 59.2° | 40 s |
| DYS EST REG2 F01 - R01 | 59.2° | 40 s |
| DYS EST REG3 F01 - R01 | 59.2° | 40 s |
| DYS EST REG4 F01 - R01 | 59.2° | 40 s |
| DYS EST REG5 F01 - R01 | 59.2° | 40 s |
| DYS EST REG6 F01 - R01 | 59.2° | 40 s |

G) Dilution and PCR

Sample dilutions were prepared for a working plate by diluting each of 19 family cohort (as described above) samples, in 0.1×TE buffer (with 0.01% Tween-20) for a final concentration in each sample of 5 ng DNA/ul, and then transferring them to new 0.5 ml tube (Nalgene). Roche human genomic DNA (Roche Diagnostics Corporation) at 5 ng/ul was used as the positive control, and 0.1×TE buffer (with 0.01% Tween-20) only was added to the negative control. Sample location/identity was preserved within the plate. PCR amplification of all regions was conducted using 10 ng of human genomic DNA and a PCR buffer containing 10 pmoles each of forward and reverse primers (Table 3), 10% 10×PCR buffer, 2 ml, MgCl₂, 2% Dimethyl Sulfoxide, 5 mM DTT, 200 uM of each dNTP, and 0.625 units of TaqGold with 1% Pfu Turbo Hotstart in a total volume of 20 ul. Reaction components were assembled in MJ Research 96-well Multiplates and briefly pulsed in a centrifuge to mix components. Cycling was performed using calculated control and a heated lid with cycles consisting of 95° C. for 12 min, followed by 35 cycles consisting of 95° C. for 30 seconds, optimal annealing temp ° C. (as selected from the gradient gel) for 20 seconds, 72° C. for appropriate extension time (Table 4), with a final extension at 72° C. for 6 min. Electrophoresis of the amplicons was performed to assess quality using 2 ul of product run on precast Nuseive/GTG 3:1 agarose gels containing ethidium bromide (BMA Corp.) and the gel image of the DYS-EST region 1 PCR showed product bands of 830 bp in length.

In order to digest excess primers and dNTPs, 5 ul of ExoSAP-IT digest mix (3.25 ul of sterile DI H2O, 1.5 ul of ExoSAP-IT (USB Corp.; Cleveland, Ohio) and 0.25 ul of 100× Acetylated Bovine Serum Albumin (Promega; Madison, Wis.) per 20 ul reaction) was then added to each well. The plates were then briefly pulsed in a centrifuge to mix components, sealed, and placed on an MJ Research thermalcycler. Cycling was performed using block control and a heated lid with cycles consisting of 37° C. for one hour, 65° C. for 10 min, and 80° C. for 10 min, followed by cooling to 4° C.

Sequence reactions were performed using 2 or 3 ul of each amplicon (depending upon electrophoretic gel band strength), 1.4 pmoles of each amplicon specific primer, and 2 ul of BigDye Terminator Ready Reactions mix version 3.00 (Applied Biosystems) per 10 ul reaction. Both forward and reverse reactions were set up for each individual primer corresponding to the primer pair employed to produce each amplicon. Reaction components were assembled in MJ Research 96-well Multiplates and briefly pulsed in a centrifuge to mix. Cycling was performed using calculated control and a heated lid with cycles consisting of 95° C. for 5 min, followed by 35 cycles consisting of 95° C. for 30 sec, 55° C. for 20 sec, and 60° C. for 4 min. Finished sequence reaction plates were pulsed in a centrifuge and 1 unit of shrimp alkaline phosphatase (USB Corp.) was added to each well. Plates were pulsed again and incubated at 37° C. for 30 min. Next, 10 ul of 10%(vol/vol) 1-Butanol in water was added to each well. Plates were pulsed to mix and samples were transferred to a Sephadex® (Sigma Chemical Co.; St. Louis, Mo. US) matrix for dye removal. The Sephadex® G50 matrix is constructed by filling the wells of a 45 ul Multiscreen® Column Loader (Millipore; Bedford, Mass. US), inverting it into a Multiscreen® Plate (Millipore), and filling each well with 300 ul De-Ionized(DI) H₂O. Before use, excess water is spun out of the plate by centrifugation at 900×g for 5 min using the S2096 rotor on an Allegra 21 Centrifuge (Beckman Coulter; Fullerton, Calif. US). After samples were transferred to the Sephadex® matrix, a MicroAmp Optical 96-well Reaction Plate (Applied Biosystems) was placed under the Sephadex® plate and the cleaned samples were collected by spinning the stack of two plates at 900×g for 5 min. The collected samples were spun in a speed vac until completely dried. 7.5 ul of De-Ionized(DI) Formamide (BioVentures, Inc.) was added to each well and the plates were cycled on a thermalcycler at 95° C. for 5 min, 80° C. for 5 min, and 4° C. for 5 min to resuspend and denature the DNA. The plates were then placed on an ABI Prisms 3700 DNA Analyzer (Applied Biosystems) using Dye Set “H, ” mobility file “DT3700Pop5(BDv3)v1.mob,” cuvette temperature 48° C., injection time 2000 seconds, and injection temperature 45° C. Sequences were then analyzed using PhredPhrap/Polyphred/Consed Suite (Codon Code Corp.; Boston, Mass. US) and Sequencher 4.1.4 (Gene Codes, Corp.; Ann Arbor, Mich.) for basecalling and contig alignment.

H) Results

The results of this procedure located a specific haplotype of microsatellite markers and various single nucleotide polymorphisms within the interval segregate with the dyslexic family members that was not found in any of the non-dyslexic family members. In this cohort, a single haplotype co-segregated with dyslexics in the effected individuals of the kindred but did not co-segregate with non-dyslexics. The inheritance of this haplotype exclusively by the related dyslexic family members confirms an autosomal dominant mode of inheritance.

### EXAMPLE 2

### Determination of a Specific Dyslexia-Related Haplotype and Single Nucleotide Polymorphisms in the Non-Related Population

A) Cohort Sample Selection

Twenty-six dyslexic samples and thirty-six non-dyslexic samples were obtained from The Center after the individuals were classified to be dyslexic using the same criteria as stated in Example 1. In the case of the non-dyslexic subjects, their non-dyslexic status was determined based on successful levels of academic achievement and personal history consistent with an absence or exclusion of dyslexia without any testing for dyslexia.

B) DNA Sample Collection and Preparation

Whole blood was collected by standard venipuncture into PAXgene blood DNA tubes (Qiagen Inc.; Valencia, Calif. US). DNA was extracted from whole blood using PAXgene Blood DNA Kit (Qiagen Inc.) according to the kit instruction handbook. Saliva samples were collected from subjects using buccal swabs and were processed similarly to obtain genomic DNA when blood could not be obtained. An additional set of 88 genomic DNAs (North American Human Variation Panel-Caucasian, Catalog No. HD100CAU) from the Coriell Institute (Camden, N.J. US) was obtained and used as a population control and were analyzed in parallel to the cohort set described above in this example.

C) Primer Selection and Optimization

The primers used for SNAJA were selected, optimized and qualified as set forth in Example 1.

D) Dilution and PCR

Sample dilutions were prepared for a working plate by diluting each of the samples in 0.1×TE buffer (with 0.01% Tween-20) for a final concentration in each sample of 5 ng DNA/ul, and then transferring them to new 0.5 ml tube (Nalgene). Roche human genomic DNA (Roche Diagnostics Corporation) at 5 ng/ul was used as the positive control, and 0.1×TE buffer (with 0.01% Tween-20) only was added to the negative control. Sample location/identity was preserved within the plate.

PCR amplification of all regions was conducted using 10 ng of human genomic DNA and a PCR buffer containing 10 pmoles each of forward and reverse primers (Table 2), 10% 10×PCR buffer (PE Biosystems), 2 mM MgCl₂ (PE Biosystems), 2% Dimethyl Sulfoxide (Sigma Aldrich; St. Louis, Mo. US), 5 mM DTT (Bio-Rad Laboratories; Hercules, Calif. US), 200 uM of each dNTP (Promega Corp), and 0.625 units of TaqGold (PE Biosystems) with 1% Pfu Turbo Hotstart (Stratagene) in a total volume of 20 ul. Reaction components were assembled in MJ Research 96-well Multiplates and briefly pulsed in a centrifuge to mix components. Cycling was performed using calculated control and a heated lid with cycles consisting of 95° C. for 12 min, followed by 35 cycles consisting of 95° C. for 30 seconds, 59.2° C. (as selected from gradient gel) for 20 seconds, 72° C. for 40 seconds, with a final extension at 72° C. for 6 min (Table 2.) Electrophoresis to assess quality of the amplicons was performed using 2 ul of product run on precast Nuseive/GTG 3:1 agarose gels containing ethidium bromide (BMA Corp.) and the gel image of the DYS-EST region 5 PCR showed product bands of 598 bp in length.

In order to digest excess primers and remaining dNTPs, 5 ul of ExoSAP-IT (USB Corp.) digest mix (3.25 ul of sterile DI H₂O, 1.5 ul of ExoSAP-IT and 0.25 ul of 100× Acetylated Bovine Serum Albumin (Promega Corp.) per 20 ul reaction) was then added to each well. The plates were then briefly pulsed in a centrifuge to mix components, sealed, and placed on the thermalcycler. Cycling was performed using block control and a heated lid with cycles consisting of 37° C. for one hour, 65° C. for 10 min, and 80° C. for 10 min followed by cooling to 4° C.

E) Sequencing and Analysis

Sequence reactions were performed using 3 ul of each amplicon, 1.4 pmoles of primer, and 2 ul of BigDye TERMINATOR® Ready Reactions mix version 3.0 (Applied Biosystems) per 10 ul reaction. Briefly, reactions were set up as in Example 1 using each PCR primer in both the forward and reverse orientation. Reaction components were assembled in MJ Research 96-well Multiplates and briefly pulsed in a centrifuge to mix. Cycling was performed using calculated control and a heated lid with cycles consisting of 95° C. for 5 min, followed by 35 cycles consisting of 95° C. for 30 sec, 55° C. for 20 sec, and 60° C. for 4 min.

Finished sequence reaction plates were pulsed in a centrifuge and 1 unit of shrimp alkaline phosphatase (USB Corp.) was added to each well. Plates were pulsed again and incubated at 37° for 30 min. Next, 10 ul of 10% 1-Butanol was added to each well. Plates were pulsed to mix and samples were transferred to a Sephadex® (Sigma Chemical Co.) matrix for dye removal. The Sephadex® matrix is constructed by filling the wells of a 45 ul Multiscreen® Column Loader (Millipore), inverting it into a Multiscreen® Plate (Millipore), and filling each well with 300 ul DI H₂O. Before use, excess water is spun out of the plate by centrifugation at 900×g for 5 min using the S2096 rotor on an Allegra 21 Centrifuge (Beckman Coulter). After samples were transferred to the Sephadex® matrix, a MicroAmp Optical 96-well Reaction Plate (Applied Biosystems) was placed under the Sephadex® plate and the cleaned samples were collected by spinning the stack of two plates at 900×g for 5 min. The collected samples were spun in a speed vacuum until completely dried. 7.5 ul of DI Formamide was added to each well and the plates were cycled on a thermalcycler at 95° C. for 5 min, 80° C. for 5 min, and 4° C. for 5 min to resuspend and denature the DNA. The plates were then placed on an ABI Prism® 3700 DNA Analyzer (Applied Biosystems) using Dye Set “H,” mobility file “DT3700Pop5(BDv3)v1.mob,” cuvette temperature 48° C., injection time 2000 seconds, and injection temperature 45° C. Sequences were then analyzed using PhredPhrap/Polyphred/Consed Suite (Codon Code Corp.) and Sequencher 4.1.4 (Gene Codes, Corp.) for basecalling and contig alignment.

Haplotype estimation was accomplished using a program from Max Delbruck Center for Molecular Medicine, Berlin at http://www.bioinf.mdc-berlin.de/projects/hap/ according to the website file format. Table 4 shows a haplotype description of the 12 SNAJA loci based on the sequence listing for SNAJA, SEQ ID NO:1. Table 7 shows the results of the SNAJA Haplotype estimation frequencies for dyslexic and non-dyslexic sample set, where 1 represents the wild type and 2 represents the positional variant.

TABLE 4
Haplotype Description of 12 SNAJA Loci Based

| | LOCUS | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| POSITION/ | A424C | C554A | A879T | C985A | G1145A | C1346T | A2275G | A2286C | G2314A | C2453T | G2613A | T3282G |
| CHANGE | | | | | | | | | | | | |
| HAPLOTYPE | 1 | 1 | 1 | 1 | 1 | 1 | 1 | 1 | 1 | 1 | 1 | 1 |
| WILD TYPE CODE | | | | | | | | | | | | |
| HAPLOTYPE | 2 | 2 | 2 | 2 | 2 | 2 | 2 | 2 | 2 | 2 | 2 | 2 |
| VARIANT CODE | | | | | | | | | | | | |

TABLE 5
Results of SNAJA Haplotype Estimation Frequencies

| | | | | GENERAL |
|---|---|---|---|---|
| | ESTIMATED | | NON- | POPU- |
| HAPLO- | 12 LOCI | DYSLEXIC | DYSLEXIC | LATION |
| TYPE | HAPLOTYPE | SAMPLES | SAMPLES | SAMPLES |
| A | 221112112121 | 0.295 | 0.250 | 0.241 |
| B | 111111111111 | 0.217 | 0.275 | 0.297 |
| C | 222112112121 | 0.200 | 0.275 | 0.300 |
| D | 111121111122 | 0.170 | 0.175 | 0.162 |
| 1 | 112111111121 | 0.083 | 0.000 | 0.000 |
| 2 | 221112122121 | 0.050 | 0.000 | 0.000 |
| 3 | 111121111121 | 0.061 | 0.000 | 0.000 |
| 4 | 221112112122 | 0.061 | 0.000 | 0.000 |
| 5 | 222112112122 | 0.050 | 0.000 | 0.000 |
| 6 | 112111111111 | 0.083 | 0.000 | 0.000 |
| 7 | 111111121121 | 0.050 | 0.000 | 0.000 |
| E | 112121211122 | 0.000 | 0.050 | 0.000 |

The finding that seven estimated 12 loci haplotypes (5, 6, 7, 8, 9, 10, 11) occurred in only the dyslexic population indicates that these haplotypes are associated with the risk of exhibiting dyslexia phenotypes, and that a variant form or forms of SNAJA is involved in the occurrence of dyslexia or is in linkage disequilibrium with another nearby genetic element contributing to dyslexia. The combined frequency for these dyslexic-associated haplotypes was found to represent 43.8% of the dyslexic cohort studies compared to the dyslexic-free sample set and the general population, a frequency substantially greater than any previously reported genetic marker associated with dyslexia.

Further, evidence of allele sharing was found using the same method for determining microsatellite alleles as described above in Example 1. The frequency of allelic sharing was found by using microsatellite sizes for each marker and their co-occurrence with one another in the general population, dyslexic and non-dyslexic samples of this Example. It was found that allele sharing of some sizes of each microsatellite have co-occurrences at an increased frequency among dyslexic samples distinct from that of non-dyslexic and general population samples as shown in Table 6.

TABLE 6
Results of Allele Sharing Frequency of D5S1487 and D5S617
for Dyslexic, Non-dyslexic and General Population Samples

| ALLELE | | | | | |
|---|---|---|---|---|---|
| COMBINATION | | | | | GENERAL |
| D5S1487 | NUMBER | NUMBER | DYSLEXIC | NON-DYSLEXIC | POPULATION |
| AND | OF | 0F | FREQUENCY | FREQUENCY | FREQUENCY |
| D5S617 | DYSLEXIC | NON-DYSLEXIC | n = 26 | n = 35 | n = 88 |
| 190,198 | 5 | 2 | 19% | 6% | 2% |
| 214,190 | 6 | 4 | 23% | 11% | 14% |
| 214,192 | 3 | 1 | 11% | 3% | 9% |

F) Results

The results of this analysis indicates that the risk of dyslexia in the sampled population was between 3.17 to 9.5 fold greater for dyslexics than non-dyslexics at the 190,198 microsatellite combination of D5S1487/D5S617; between 1.64 to 2.09 fold greater for dyslexics than non-dyslexics at the 214,190 microsatellite combination of D5S1487/D5S617; and between 1.2 to 3.67 fold greater for dyslexics than non-dyslexics at the 214,192 microsatellite combination of D5S1487/D5S617. Therefore, these markers D5S1487/D5S617 can be utilized by themselves, in combination with one another or used in combination with other markers on other chromosomes to evaluate epistatic interactions between genes in order to classify populations, families or individuals for risk of occurrence of dyslexia.

As will be understood by those with skill in the art with reference to this disclosure, the gene, SNAJA, which is expressed only in the brain, exhibits variant haplotypes which associate with dyslexia and are absent from the non-dyslexic cohort and the North American Caucasian population control group. Further, these findings are especially striking considering the finding that >40% (43.8%) of the non-related dyslexic cohort have variant genetic forms of the genomic sequence of SNAJA, SEQ ID NO:1, which are absent from both the population control group and the non-dyslexic cohort examined in this Example 2.

Therefore, as disclosed in this disclosure, according to one embodiment of the present invention, there is provided two previously unknown single nucleotide polymorphisms present within SEQ ID NO:1 on Chromosome 5 that can be used to indicate the presence of dyslexia or a predisposition to develop dyslexia. Therefore, in one embodiment, the present invention is an isolated polynucleotide comprising at least about 17 consecutive nucleotides of SEQ ID NO:1 including residue 2285, where residue 2286 has an A to C substitution. In another embodiment, the present invention is a polynucleotide comprising at least about 17 consecutive nucleotides of SEQ ID NO:1 including residue 3281, where residue 3282 has a T to G substitution. Therefore, in one embodiment, the present invention is a polynucleotide comprising at least about 25 consecutive nucleotides of SEQ ID NO:1 including residue 2285, where residue 2286 has an A to C substitution. In another embodiment, the present invention is a polynucleotide comprising at least about 25 consecutive nucleotides of SEQ ID NO:1 including residue 3281, where residue 3282 has a T to G substitution. Therefore, in one embodiment, the present invention is a polynucleotide comprising at least about 40 consecutive nucleotides of SEQ ID NO:1 including residue 2285, where residue 2286 has an A to C substitution. In another embodiment, the present invention is a polynucleotide comprising at least about 40 consecutive nucleotides of SEQ ID NO:1 including residue 3281, where residue 3282 has a T to G substitution.

According to one embodiment of the present invention, there are provided seven haplotypes on Chromosome 5 that indicate the presence of dyslexia or a predisposition to develop dyslexia. These seven haplotypes are:
Haplotype #1: A879T and G2613A variants of SEQ ID NO:1 in combination;
Haplotype #2: A424C, C554A, C1346T, A2286C, G2314A and G2613A variants of SEQ ID NO:1 in combination;
Haplotype #3: G1145A and G2613A variants of SEQ ID NO:1 in combination;
Haplotype #4: A424C, C554A, C1346T, G2314A, G2613A and T3282G variants of SEQ ID NO:1 in combination;
Haplotype #5: A424C, C554A, A879T, C1346T, G2314A, G2613A and T3282G variants of SEQ ID NO:1 in combination;
Haplotype #6: A879T variant of SEQ ID NO:1 in combination; and
Haplotype #7: A2286C and G2613A variants of SEQ ID NO:1 in combination.

Therefore, in one embodiment, the present invention is isolated genetic material from human Chromosome 5 that indicates the presence of dyslexia or a predisposition to develop dyslexia in the individual from whom the sample was obtained, the material comprising a variant of SEQ ID NO: 1 comprising (Haplotype #1) an A to T substitution at residue 879 and a G to A substitution at residue 2613; or comprising (Haplotype #2) an A to C substitution at residue 424, a C to A substitution at residue 554, a C to T substitution at residue 1346, an A to C substitution at residue 2286, a G to A substitution at residue 2314 and a G to A substitution at residue 2613; or comprising (Haplotype #3) a G to A substitution at residue 1145 and a G to A substitution at residue 2613; or (Haplotype #4) comprising an A to C substitution at residue 424, a C to A substitution at residue 554, a C to T substitution at residue 1346, a G to A substitution at residue 2314, a G to A substitution at residue 2613 and a T to G substitution at residue 3282; or comprising (Haplotype #5) an A to C substitution at residue 424, a C to A substitution at residue 554, an A to T substitution at residue 879, a C to T substitution at residue 1346, a G to A substitution at residue 2314, a G to A substitution at residue 2613 and a T to G substitution at residue 3282; or comprising (Haplotype #6) an A to T substitution at residue 879; or comprising (Haplotype #7) an A to C substitution at residue 2286 and a G to A substitution at residue 2613; where except for these substitutions, residue 424 is A, residue 554 is C, residue 879 is A, residue 985 is C, residue 1145 is G, residue 1346 is C, residue 2275 is A, residue 2286 is A, residue 2314 is G, residue 2453 is C, residue 2613 is G, residue 3282 is T. As will be understood by those with skill in the art with reference to this disclosure, other residues of SEQ ID NO:1 will vary between individuals and populations and these variations do not change the invention disclosed in this disclosure. The claimed subject matter is considered to encompass these other variations.

According to one embodiment of the present invention, there are provided three haplotypes on Chromosome S that indicate the presence of dyslexia or a predisposition to develop dyslexia. Each haplotype comprises an allele of each of at least two microsatellite markers flanking SNAJA, SEQ ID NO: 1, in combination on Chromosome 5 are Haplotype #8 the 190,198 microsatellite combination of D5S1487/D5S617; Haplotype #9 the 214,190 microsatellite combination of D5S1487/D5S617; and Haplotype #10 the 214,192 microsatellite combination of D5S1487/D5S617.

According to another embodiment of the present invention, there is provided a method of diagnosing dyslexia or a predisposition to develop dyslexia. In one embodiment, the method comprises, first, providing a sample from an individual containing genetic material from Chromosome 5. In one embodiment, the sample is analyzed for the presence of one or more than one of Haplotype #1 through Haplotype #10, where the presence of one or more than one of Haplotype #1 through Haplotype #10 indicates a diagnosis of dyslexia or a predisposition to develop dyslexia. In another embodiment, the sample is analyzed for the presence of one or more genetic variant that decreases the amount or activity of the gene product of the SNAJA gene, SEQ ID NO: 1, as compared with the amount of the gene product or the amount of gene product activity for non-dyslexics, where the presence of the variant of the gene indicates a diagnosis of dyslexia or a predisposition to develop dyslexia. In one embodiment, the sample is analyzed by contacting the sample with a polynucleotide probe complimentary to the mRNA of a variant form of SNAJA, SEQ ID NO:1, known to produce a decreased amount of gene product or a gene product having decreased activity. In one embodiment, the sample is obtained in utero or post-mortem, rather than from a living individual post birth.

According to another embodiment of the present invention, there is provided a method of diagnosing dyslexia or a predisposition to develop dyslexia. The method comprises, first, providing a sample from an individual potentially containing a gene product of SNAJA, SEQ ID NO:1. Next, the sample is analyzed to determine the amount or activity or both of the gene product of SNAJA, SEQ ID NO:1, where the presence of a decreased amount or activity or both of the gene product indicates a diagnosis of dyslexia or a predisposition to develop dyslexia. In one embodiment, the sample is analyzed by contacting the sample with antibodies to the gene product of SNAJA, SEQ ID NO:1. In a preferred embodiment, the gene product is selected from the group consisting of SEQ ID NO:10 and SEQ ID NO:11. To distinguish between the two gene products, antibodies should be directed to the carboxy terminus of each as this is where they have maximal differences between them. In a preferred embodiment, the antibodies are directed specifically to the last 6-8 amino acids of the carboxy termini or either SEQ ID NO:10 or SEQ ID NO:11 or both. In one embodiment, the sample is obtained in utero or post-mortem, rather than from a living individual post birth.

The methods of the present invention can additionally comprise administering phonological testing to the individual to confirm the diagnosis of dyslexia. The method can additionally comprise analyzing genetic material from the individual for the presence of one or more than one genetic marker for dyslexia or for a predisposition to develop dyslexia on a chromosome other than Chromosome 5 to confirm the diagnosis of dyslexia. In a preferred embodiment, the chromosome other than Chromosome 5 is selected from the group consisting of Chromosomes 1p, 2p, 3p, 3q, 4q, 6p21.3, 6q, 8p, 9p, 11p, 13q, 15q, 18p, 18q, 21q, and Xq. In a particularly preferred embodiment, the chromosomes other than Chromosome 5 are Chromosomes 6p21.3 and 18p11.2.

According to another embodiment of the present invention, there is provided a kit for diagnosing dyslexia or a predisposition to develop dyslexia. The kit comprises one or more than one primer identified in Table 1, that is SEQ ID NO:2 through SEQ ID NO:9 designed to identify the presence of a polynucleotide according to the present invention, or the presence of one or more than one Haplotype #1 through Haplotype #10, or a combination of the preceding. In a preferred embodiment, the kit comprises all of the primers identified in Table 1, that is SEQ ID NO:2 through SEQ ID NO:9. As will be understood by those with skill in the art with reference to this disclosure, the term “primer” as used in context with the kit of the present invention is intended to include polynucleotide sequences longer or shorter than the exact sequences given in Table 1, such as between 1 and 5 nucleotides shorter, and between 1 and 10 nucleotides longer suitable for amplifying SEQ ID NO:1. The kit can further comprise one or more than one agent, substance or material selected from the group consisting of a PCR buffer, a thermostable DNA polymerase and dNTPs.

According to another embodiment of the present invention, there is provided a method of ameliorating the symptoms of dyslexia or preventing dyslexia. The method comprises diagnosing dyslexia or a predisposition to develop dyslexia in an individual using a method according to the present invention, and then treating the individual. In one embodiment, treating the individual comprises administering phonological training to the individual. In another embodiment, treating the individual comprises administering to the individual an amount of the gene product of the SNAJA gene in sufficient quantities to compensate for the missing or non-functional gene product due to the presence of the individual's genetic variants in the SNAJA gene. In another embodiment, treating the individual comprises administering to the individual an amount of one or more than one pharmaceutical agent employed to treat cognitive or emotional disorders which demonstrate association or overlap with the dyslexic phenotype. In a preferred embodiment, the pharmaceutical agent is a lithium salt. In another embodiment, the pharmaceutical agent is carbamazipine. As will be understood by those with skill in the art with reference to this disclosure, the dose, route and frequency of administration is within the knowledge of one of ordinary skill, and can be determined using standard sources, such as for example, Physician Desk Reference 2002, 57^{th} Edition, Medical Economics Company, Montvale, N.J. US.

According to another embodiment of the present invention, there is provided a method of classifying a dyslexic individual or individuals comprising, first, diagnosing dyslexia or a predisposition to develop dyslexia in the individual or individuals according to the method of the present invention, and then, assigning a classification to the individual or individuals based on the variant or haplotype identified as a result of the diagnosis.

Every reference cited in this disclosure is hereby incorporated herein by reference in its entirety.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

What is claimed is:

1. Isolated genetic material from human Chromosome 5 of an individual that indicates the presence of dyslexia or a predisposition to develop dyslexia in the individual from whom the material was obtained, the material comprising an allele of each of at least two microsatellite markers flanking SEQ ID NO:1 in combination on Chromosome 5: Haplotype #8 the 190,198 microsatellite combination of D5S1487/D5S617; Haplotype #9 the 214,190 microsatellite combination of D5S1487/D5S617; and Haplotype #10 the 214,192 microsatellite combination of D5S1487/D5S617.

2. Isolated genetic material from human Chromosome 5 of an individual that indicates the presence of dyslexia or a predisposition to develop dyslexia in the individual from whom the material was obtained, the material comprising:
a) isolated genetic material according to claim 1; in combination with either
b) an isolated polynucleotide comprising at least about 17 consecutive nucleotides of SEQ ID NO:1 including residue 2285, where residue 2286 has an A to C substitution; or comprising at least about 17 consecutive nucleotides of SEQ ID NO:1 including residue 3281, where residue 3282 has a T to G substitution; or comprising at least about 25 consecutive nucleotides of SEQ ID NO:1 including residue 2285, where residue 2286 has an A to C substitution; or comprising at least about 25 consecutive nucleotides of SEQ ID NO:1 including residue 3281, where residue 3282 has a T to G substitution; or comprising at least about 40 consecutive nucleotides of SEQ ID NO:1 including residue 2285, where residue 2286 has an A to C substitution; or comprising at least about 40 consecutive nucleotides of SEQ ID NO:1 including residue 3281, where residue 3282 has a T to G substitution; or
c) isolated genetic material from human Chromosome 5 of an individual that indicates the presence of dyslexia or a predisposition to develop dyslexia in the individual from whom the material was obtained, the material comprising a sufficient portion of SEQ ID NO:1 comprising (Haplotype #1) an A to T substitution at residue 879 and a G to A substitution at residue 2613; or comprising (Haplotype #2) an A to C substitution at residue 424, a C to A substitution at residue 554, a C to T substitution at residue 1346, an A to C substitution at residue 2286, a G to A substitution at residue 2314 and a G to A substitution at residue 2613; or comprising (Haplotype #3) a G to A substitution at residue 1145 and a G to A substitution at residue 2613; or (Haplotype #4) comprising an A to C substitution at residue 424, a C to A substitution at residue 554, a C to T substitution at residue 1346, a G to A substitution at residue 2314, a G to A substitution at residue 2613 and a T to G substitution at residue 3282; or comprising (Haplotype #5) an A to C substitution at residue 424, a C to A substitution at residue 554, an A to T substitution at residue 879, a C to T substitution at residue 1346, a G to A substitution at residue 2314, a G to A substitution at residue 2613 and a T to G substitution at residue 3282; or comprising (Haplotype #6) an A to T substitution at residue 879; or comprising (Haplotype #7) an A to C substitution at residue 2286 and a G to A substitution at residue 2613; where except for these substitutions, residue 424 is A, residue 554 is C, residue 879 is A, residue 985 is C, residue 1145 is G, residue 1346 is C, residue 2275 is A, residue 2286 is A, residue 2314 is G, residue 2453 is C, residue 2613 is G, residue 3282 is T; or
d) both b) and c).

3. A method of diagnosing dyslexia or a predisposition to develop dyslexia, the method comprising:
a) providing a sample from an individual containing genetic material from Chromosome 5; and
b) analyzing the genetic material for the presence of one or more than one of Haplotype #8 through Haplotype #10 according to claim 1, or isolated genetic material according to claim 2;
where the presence of one or more than one of Haplotype #8 through Haplotype #10 according to claim 1 or isolated genetic material according to claim 2 indicates a diagnosis of dyslexia or a predisposition to develop dyslexia.

4. The method of claim 3, where the sample is obtained in utero or post-mortem.

5. The method of claim 3, additionally comprising administering phonological testing to the individual to confirm the diagnosis of dyslexia.

6. The method of claim 3, additionally comprising analyzing genetic material from the individual for the presence of one or more than one genetic marker for dyslexia or for a predisposition to develop dyslexia on a chromosome other than Chromosome 5 to confirm the diagnosis of dyslexia.

7. The method of claim 6, where the chromosome other than Chromosome 5 is selected from the group consisting of Chromosomes 1p, 2p, 3p, 3q, 4q, 6p21.3, 6q, 8p, 9p, 11p, 13q, 15q, 18p, 18p11.2, 21q, and Xq.

8. The method of claim 6, where the chromosome other than Chromosome 5 is selected from the group consisting of Chromosomes 6p21.3 and 18p11.2.

9. A method of ameliorating the symptoms of dyslexia or preventing dyslexia in an individual, the method comprising:
a) diagnosing dyslexia or a predisposition to develop dyslexia in the individual according to the method of claim 3; and
b) treating the individual.

10. The method of claim 9, where treating the individual comprises administering phonological training to the individual.

11. A method of classifying a dyslexic individual or group of dyslexic individuals comprising:
a) diagnosing dyslexia or a predisposition to develop dyslexia in the individual or individuals according to the method of claim 3; and
b) assigning a classification to the individual or individuals based on the variant or haplotype identified as a result of the diagnosis.
